(19) United States
(12) Patent Application Publication
(10) Pub. No.: US 2018/0229048 A1
(43) Pub. Date: Aug. 16, 2018

(54) METHOD AND DEVICE FOR BODY FLUID STIMULATION

(71) Applicant: BTL Holdings Limited, Nicosia (CY)

(72) Inventors: Jan Sikora, Frydek-Mistek (CZ); Tomás Schwarz, Prague (CZ)

(21) Appl. No.: 15/433,210

(22) Filed: Feb. 15, 2017

(51) Int. Cl. A61N 2/00; A61N 7/00; A61H 23/00; A61N 1/44; A61N 1/36; A61N 1/40

(52) U.S. Cl. CPC A61N 2/002; A61N 7/00; A61H 23/00; A61H 2201/10; A61N 1/36003; A61N 1/36014; A61N 1/40; A61N 1/44

(57) ABSTRACT

A method of soft tissue treatment of a patient includes placing a treatment sleeve with at least one first treatment energy source onto the patient. Massage of the patient's soft tissue is provided by at least one massage unit, with the massage unit including at least two pressure changing elements applied on the patient's soft tissue. An additional or second treatment therapy, different from the massage, is provided by a second treatment energy source. The massage unit creates a pressure gradient between at least two successive elements changing a pressure value in range from 0% to 95% on the patient's soft tissue across a treated body area and stimulates body liquid flow.

### FIELD OF THE INVENTION

The field of the invention relates to apparatus and automated methods for aesthetic treatment using a combination of lymph drainage and radio-frequency (RF) providing soft tissue treatment.

### BACKGROUND OF THE INVENTION

Human skin is tissue which is commonly treated in order to improve its visual appearance. Skin is composed of three basic elements: the epidermis, the dermis and the hypodermis or so called subcutis. The outer and also thinnest layer of skin is the epidermis. Epidermis contains mainly stratified squamous epithelium of which the outer side keratinizes and ensures coverage whereas the inner side contains a pigment. The dermis consists of collagen, elastic tissue and reticular fibers. The hypodermis is the lowest layer of the skin and contains hair follicle roots, lymphatic vessels, collagen tissue, nerves and also fat forming a subcutaneous white adipose tissue (SWAT).

SWAT is formed by aggregation of fat cells ranging around 50 microns and more in diameter and containing as much as 95% glycerides and fatty acids by volume. Overeating and unhealthy lifestyles may result in an increase of size and/or number of the fat cells. The fat cells create lobules which are bounded by connective tissue, fibrous septa (retinaculum cutis). The protrusions of these lobules are also knows as cellulite.

Another part of adipose tissue located in peritoneal cavity is known as abdominal obesity. The visceral fat layer forming visceral white adipose tissue (VWAT) is located between the parietal peritoneum and the visceral peritoneum, closely below muscle fibers adjoining the hypodermis layer.

Excess adipose tissue in the subcutaneous or abdominal area may be perceived as aesthetically undesirable, mainly in the buttocks, thighs, abdomen or hips, where even weight loss after dieting and exercise may not lead to satisfactory results. Moreover, in the last few decades, more people suffer from growth of visceral white adipose tissue (VWAT) mainly in their abdominal area. Visceral fat has been linked to various cardiovascular diseases and diabetes.

The undesirable topographic skin and/or body appearance may also be caused by changes in the dermal or sub-dermal layer of the skin, especially by excessive numbers or volume of fat cells, weakening of fibrous septas, loss of elasticity and/or limited lymph and blood circulation that may result in swelling, accumulation of toxins and/or other visually and/or health undesired effects. Furthermore, pigment inhomogeneity caused by structural changes in epidermis and/or by pigment in the skin may also lead to low self-confidence of people with this skin condition.

Toxic, undesired substances, damaged cells and/or excess of some substances are removed from the soft tissue by lymphatic system and blood circulation system. Blood and optimal lymphatic circulation are crucial for safe and high effective treatment (e.g. prevention of panniculitis) and healthy life. Stimulation of the lymphatic system may help to reduce swelling, improve immune system reactions, improve metabolism and function of patient's cells and/or may leach undesirable substances from the soft tissue and contribute to filtration and metabolism.

Nowadays, blood and/or lymph circulation may be improved by several methods e.g. by massage, applied pressure, heating, cooling and other methods. However, these methods require manual guiding or separate therapies, which may be very time consuming. Further, these methods require a trained practitioner and may be expensive.

No devices or methods in the known current state of art are able to provide automatic systematic large body area massage and/or lymph drainage in combination with adapted RF energy delivering into the soft tissue. There are no devices or methods which stimulate the blood and/or lymph flow with continual treatment by electromagnetic, acoustic wave, shock wave or plasma energy in order to provide effects described below. There is also need to improve the state of art in order to provide self-operated devices, prevention of human mistakes and large area treatment with better results even in the most problematic parts of human body.

### SUMMARY OF THE INVENTION

Apparatus and methods provide massage treatment with positive pressure (higher than atmospheric pressure), negative pressure (lower than atmospheric pressure), vibration and/or other massaging treatment therapy (e.g.: by magnetic muscle stimulation, electrostimulation of the muscle and/or massaging of the soft tissue by acoustic waves) in combination with at least one more specific treatment energy source delivered to the patient's soft tissue: radio-frequency (RF) treatment energy source, magnetic field source, acoustic wave treatment energy source, shock wave treatment energy source, light energy source, plasma source, thermal energy source, mechanical pressure treatment energy source and/or other treatment energy source directed to the patient's soft tissue to provide specific treatment therapy. These types of energies may be delivered into the treated soft tissue of the patient in a sequential or simultaneous manner.

The apparatus may provide preheating, heating, precooling and/or cooling of the soft tissue using any treatment energy source and/or by thermal diffusion between objects of different temperature. Changing temperature of the soft tissue in the beginning, during and/or at the end of the treatment may influence pain receptors, soft tissue laxity, dielectric properties, improve homogeneity of distributed energy delivered by a treatment energy source (e.g. prevent hot spots), stimulate fat metabolism, prevent edge effects, and/or create a thermal gradient in the soft tissue. A temperature of the soft tissue target area during the treatment may be may be selectively adjusted with or without changing temperature of adjacent areas, in order to improve comfort and/or effectiveness of the treatment.

Changing temperature of the soft tissue before, during and/or after treatment may be provided by thermal diffusion from a warmer object (and/or substance e.g. air, liquid) and/or by thermal radiation, light waves, RF waves, acoustic waves, plasma, mechanical friction and/or by another treatment energy source. Various aesthetic skin and/or body treatment therapies may be provided by the present methods and devices including: anti-ageing (e.g.: wrinkle reduction, skin tightening, hydrating the skin, skin rejuvenation, skin viability, removing of pigment deficiencies and/or pigmentation correction); skin disease (e.g.: rashes, lupus, fungal diseases, surface antimicrobial treatment procedure, hypothermia, hyperaemia); soft tissue relaxation (e.g.: muscle and/or other soft tissue layers relaxation); body shaping (e.g.: fat removing, removing of unwanted soft tissue laxity, removing of cellulite, building muscle mass and strength, accelerating fat metabolism of a cells, restructuring of the connective tissue; increase in the number of fibroblasts, start up neocolagenesis and/or elastogenesis); and/or other soft tissue deficiencies (e.g.: accelerating body metabolism, stimulating the lymphatic circulation), influence membrane transport of a cell, a proliferation of chondrocytes in the cartilages, increase in blood profusion, blood flow and venous return, wound healing, disinfection of the patient surface and/or relieving of a patient body pain); analgesic effects resulting from specific frequencies of the delivered signal influencing on nerves.

In one aspect the device may be shaped to provide better contact with the patient's body, for example in the shape of a compression bag, sleeve, trousers, shorts, a shirt, jacket or other garment. The device may treat any one or multiple body parts. In some embodiments the device may enable treatment of multiple body parts at the same time. It may be also used more than one design embodiment (e.g. sleeve) at the same time. One or more design embodiments may treat individually and/or may be interconnected and may cooperate. Massage units may be designed in order to provide lymph drainage.

Massage may be provided by one or more static massage units and/or one or more elements changing pressure values to the patient's soft tissue. One or more elements changing pressure values to the patient's soft tissue may do translational and/or rotational moves in one, two and/or three Cartesian coordinates. A massage unit includes one or more elements changing pressure value to the patient's soft tissue in the meaning that this massage unit applies different pressure to patient soft tissue. Several embodiments of massage unit may create a pressure gradient in the soft tissue without touching the skin. Such pressure gradients may be targeted on the soft tissue layer, under the skin surface and/or to different soft tissue structure. Massage units may contain pressure cells, massage object described in FIG. 3, or other embodiments of elements changing pressure value.

Massage may also be provided by a massage unit that provides contraction/relaxation and/or pressure stimulation of one or more layers of the soft tissue by other ways besides applying external pressure to the patient body. Such source of massage may be e.g. muscle stimulation by electric, magnetic field and/or may be created pressure gradients by temperature gradients, applying waves based on acoustic waves, shock waves etc. In some embodiments massage may also be provided by accelerating one or more massaging objects described below. In some other embodiment may be used implemented thermal, magnetic and/or electrical shape changing materials. Accelerated massaging may be provided by flow of one or more a gaseous and/or liquid substances), electric field, magnetic field and/or combination thereof.

Massage accelerates and improves treatment therapy by RF (and/or other therapy that influence cell metabolism, disrupts cell wall and/or change continuity of some parts of the soft tissue) improves blood and/or lymph circulation, anti-edema, erythema effect, accelerates removing of the fat, accelerate metabolism, accelerates elastogenesis and/or neocolagenesis.

Predefined treatment therapy (procedure) may be manual (by operator) and/or automatically changed during the treatment based on feedback parameters and/or based on the treatment protocol before the treatment based on previous treatment or measurement, predefined treatment protocol and/or according experience of the operator.

Multiple treatment energy sources and/or therapies may be combined to provide a synergic effect on human tissue. This improves effectivity of the treatment and/or reduces time needed for the treatment. It may also improve safety of the treatment e.g. stimulation of soft tissue by massage improves blood and lymph stimulation which in combination with RF field provides improved removal of treated fat cells (prevention of panniculitis), improved homogeneity of delivered energy in to the soft tissue, targeting of delivered energy to the soft tissue, reduced pain during the therapy and/or a decrease influence of edge effect and overheating of a part of the soft tissue due to better body liquid circulation. Another example of synergic effect may be using plasma, where an RF electrode may regulate, help to create plasma and/or adjust some parameters of plasma. Several therapies in combination may provide better transfer of the energy to a specific layer of the soft tissue; e.g. preheating, massage of the patient surface to accelerate blood flow that increase complex dielectric constant of surface and increases penetration of such layer by RF waves.

The apparatus may operate without any operator which saves time and reduces costs of the treatment. The apparatus may automatically control a treatment sleeve and/or one or more treatment energy sources. One operator may supervise more than one treated patient. Self-operated devices may prevent mistakes during the treatment caused by human factors; a self-operated device may also have a better response to changed conditions of the treatment and/or may provide more homogenous and precise treatment which improves results and safety of the treatment. A computer has better response to changed conditions because it can react faster than 0.01 s, whereas human response on occurrences like moving of the patient, or structural changes in the soft tissue is at least 0.5 s. Another benefit of self-operated device may be that operator does not have to be as skilled as when using manual device.

Systematic, continuous, large body area massage and application of the other different therapy e.g. RF field therapy that may provide more effective treatment. Pressure provided to the soft tissue improves effectiveness of the delivering RF signal to the soft tissue and possibility to treat specific components of the soft tissue selectively and with better results. Treatment is not limited by two hands of the operator.

Embodiments may be flexible and can be adapted to patient surface of arbitrary size and shape. This characteristic helps to provide optimal energy transfer from an applicator to the patient soft tissue. Perfect contact with the patient surface may decrease or prevent the edge effect, backscattering of delivered energy may improve focusing of the delivered energy and/or provides optimal conditions for collecting feedback information. Direct contact with the patient surface may also be used for accurate and fast regulation of patient surface temperature.

The device and/or method allows an operator to arbitrary choose several treatment therapies and procedures that may work simultaneously, with some overlay and/or sequentially and select and/or adjust parameters of the procedure before the treatment and/or during the treatment without the need of continual control, operation or supervision of the device.

### BRIEF DESCRIPTION OF THE DRAWINGS

FIG. 1 is a schematic diagram of a treatment device.

FIG. 2 illustrates influence of pressure on lymph and blood circulation.

FIG. 3 illustrates several embodiments (a-f) of static massage units.

FIG. 4 is a cross-section of a treatment sleeve and its layers.

FIG. 5 illustrates a treatment sleeve with displayed treatment feedback information.

FIG. 6 shows use of several massage units.

FIG. 7 illustrates treatment/massage patterns.

FIG. 8 illustrates a massage unit that provides vibrations.

### GLOSSARY

Soft tissue includes skin, muscle, fat, fibrous tissue, nervous tissue (e.g. neurons, motor neuron and neuromuscular junction).

Patient is a human or an animal and/or limbs, tissue, sections or areas of them. Skin is composed of three basic elements: the epidermis, the dermis and the hypodermis or so called subcutis. Patient surface generally is the skin surface (epidermis).

Operator is a knowledgeable person who is authorized to perform treatment by the present methods and devices. Treatment protocol is a software protocol that defines treatment parameters and guide treatment process of one or more therapy units and guiding of its parameters. Treatment protocol may be preprogrammed in a control unit, may be used external treatment protocol and/or treatment protocol may be adjusted by operator, defined by operator and/or saved in a control unit (e.g.: downloaded from a network, recorded from an external device, implemented during the production etc.). Two or more protocols may be composed together and create one new treatment protocol.

A treatment unit includes a massage unit and/or other unit that generates treatment energy source (e.g.: RF treatment unit, acoustic wave treatment unit, shock wave treatment unit, mechanical wave treatment unit, treatment unit applied magnetic field, electric current, electric field, coherent electromagnetic radiation, non-coherent electromagnetic radiation, plasma source). A treatment sleeve may include one or more massage units. A massage unit is type of treatment unit that provides massaging effects e.g. relaxation of the soft tissue, lymph drainage and/or body liquid flow stimulation. Massaging effects may be provided by elements changing pressure and/or treatment energy sources.

Muscle and adjacent soft tissue relaxation and/or body liquid flow stimulation may be provided by positive or negative mechanical pressure provided by a suction mechanism, airflow, liquid flow, pressure provided by an object (e.g. massaging object, pressure cells etc.) and/or in other ways. Massage may be provided by muscle stimulation by electric, magnetic and/or electromagnetic field treatment energy source.

Massage may be also provided by acoustic wave, ultrasound wave, shock wave treatment energy source creating pressure gradient in the soft tissue.

Massage may also be provided by a low intensity RF treatment energy source scanning target area to heat up body liquid fluid and/or other soft tissue structures in order to provide body liquid fluid stimulation and soft tissue relaxation.

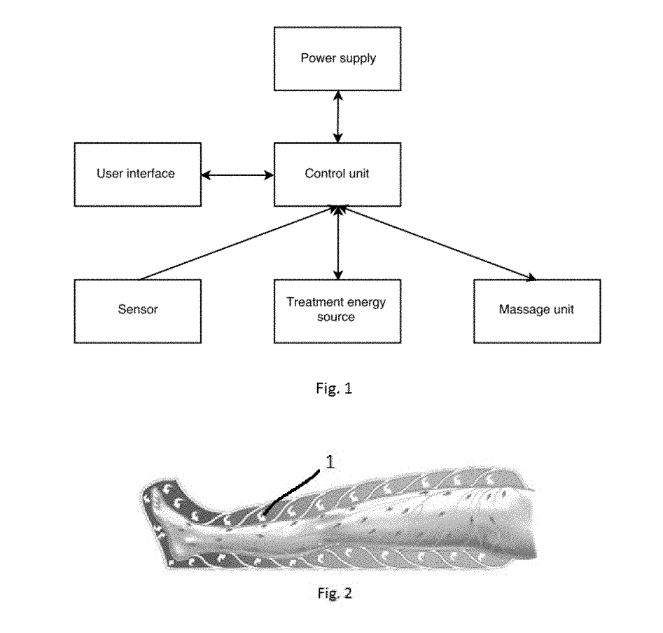

Massage may also be provided by different treatment energy sources and/or mechanisms. Massage unit provide a massaging effect. Massage units may include one or more treatment energy sources providing massage wherein the one or more treatment energy sources may include at least one element changing pressure value (e.g. massage object, pressure cell and/or other) on and/or in the soft tissue and providing a massaging effect. One massage unit may also include one or more elements changing pressure provided to the patient's soft tissue (e.g. one massage unit may include more than one pressure cells, as shown in FIG. 2). An element changing pressure provided to the patient's soft tissue is an element of the massage unit that is a source of the massaging effect (e.g. massaging object, pressure cell, a source of energy or of electric current providing muscle stimulation).

Treatment therapy distinguishes a treatment energy source, mechanisms of influencing target soft tissue and target type of soft tissue. Treatment therapy providing massage by electro stimulation of muscle fiber is different than massage provided by mechanical pressure. Also RF heating of collagen fibers and fat tissue in order to remove cellulite is another type of the treatment therapy than massage or removing fat by shock wave treatment or removing fat by RF without significant heating of collagen fibers. Treatment therapy may be e.g.: massage provided by mechanical pressure, massage by electro muscle stimulation, massage by magnetic field muscle stimulation, massage by acoustic wave, antibacterial plasma treatment, heating by thermal diffusion, removing of fat by light, removing of fat by RF, removing of fat by shock wave, wrinkle reduction by RF, wrinkle reduction by shock wave, preheating by RF and many others. Different treatment therapies using the same treatment energy source may have different results e.g. one RF treatment therapy may be used to reduce wrinkles and the other treatment therapy may be used to reduce belly fat.

Body fluid/liquid stimulation means lymphatic system stimulation and/or blood circulation stimulation.

Active treatment/massage means providing at least one type of treatment energy source (including providing pressure to the patient's body) in order to provide at least one type of specific treatment therapy e.g.: massage, RF treatment therapy, light therapy and/or others. The treatment energy source has defined parameters according to a treatment protocol and in some embodiments and/or according some treatment protocols these parameters may be changed during the treatment.

A treatment sleeve layer may be any part of the treatment sleeve from any kind of material. Layers of the treatment sleeve also includes any substances located between the treatment sleeve and patient's skin. The substances between the treatment sleeve and patient's skin may be on a liquid base, gel base, suspension base and/or solid base (e.g. any type of clothes on the patient's skin.)

### DETAILED DESCRIPTION

The devices and methods may have several embodiments similar in appearance e.g.: a compression bag, sleeve, trousers, short, shirt, jacket and/or other part of a clothes and/or other covering of a patient body part that may or may not encircle whole patient limb and/or body. One embodiment may cover only part of the patient's limb and/or body part.

A treatment sleeve may provide massage and/or other treatment therapy provided by one or more specific treatment energy sources adjacent to the patient soft tissue. Treatment energy source adjacent to the patient or to the patient's soft tissue means that the treatment energy source is in direct contact with the patient's skin, in indirect contact with the patient's skin (the treatment energy source is spaced by any layer of the treatment sleeve and/or clothes) and/or no-contact with the patient's skin (treatment energy source is spaced of the patient's skin by air or in some embodiment is spaced by air and any other layer of the treatment sleeve).

The devices and methods may be applied to different body parts e.g.: arm, leg, buttocks, hips, torso, belly and/or other body part. The device and methods can treat large body areas. The treated area during one or more treatment protocol may be in range 1 cm² to 3 m² in more preferred embodiment in range between 50 cm² to 2 m² in the most preferred embodiment in range between 100 cm² to 1.5 m².

The device may provide multiple types of the treatment therapies by different treatment energy sources e.g.: massage and/or other pressure therapy, RF-therapy, thermal therapy, light (coherent and/or non-coherent light), electrotherapy, therapy by magnetic field, acoustic wave therapy, shock wave therapy and/or therapy by plasma. Some of the treatment energy sources mentioned above may be used in combination simultaneously, with overlay, sequentially and/or separately. Parameters, combinations and/or sequences of treatment energy sources may produce different treatment therapies (e.g. used frequencies, intensity and/or focusation of RF treatment energy source may lead to wrinkle reduction therapy or on the other hand may lead to fat reduction).

As shown in FIG. 1 treatment of a patient may be guided by a control unit according to one or more predefined treatment protocols and/or according parameters set in a user interface where an operator may set parameters of treatment protocol. The operator may also design new treatment protocol and/or set up sequences of multiple treatment protocols. A sequence of several treatment protocols composed together creates a new treatment protocol. When an operator sets treatment parameters, the rest of the treatment parameters may be automatically calculated, remain as originally set and/or may be changed according predefined protocol in order to provide safe treatment.

In some embodiments the treatment protocol may be set and/or modified automatically based on treatment history of a specific patient, measurement provided on the patient body and/or feedback information received before treatment and/or during the treatment.

In another embodiment the system may be able to learn from earlier treatments and/or during the treatment. Such an operating system may be able to optimize the treatment process based on feedback information and/or based on information loaded from external device.

In some embodiments treatment may be guided by more than one control unit. A control unit may be located in the treatment sleeve and/or in an external device. A first control unit may guide a treatment sleeve and a second control unit may be located in another device such as described in U.S. Pat. No. 8,548,599, incorporated herein by reference, where these two or more control unit may cooperate.

The present devices and methods may be automated, i.e., providing treatment without need of an operator, or with partial operator participation (e.g. operator setting and/or adjusting some of treatment parameters before and/or during the treatment).

Treatment parameters may be any parameters related to the treatment of the patient (e.g. a value of applied pressure, switching on/off sequence of specific treatment energy sources, therapies, intensity of delivered treatment energy into the patient's soft tissue, pulse duration, pulse sequence, impulse duration, frequency of delivered energy by treatment energy source, amount of delivered radiation, operation time of the treatment therapy, timing of applied therapies, placement of specific layer(s) in the treatment sleeve, placement of individual treatment energy sources and/or sensors, treatment temperature on the patient surface and/or in the patient's soft tissue, focusation parameters of delivered energy to the soft tissue (e.g. focal spot size, number of focus, location of treatment energy focus, delivered energy is focused or nonfocused and/or other focusation parameters) value of electric/magnetic stimulation, rate of increasing/decreasing applied pressure value, location of applied therapy and/or other parameters).

Treatment parameters may be modified automatically by the control unit and/or by the operator based on a value of one or more parameters and/or based on a warning displayed on the user interface. Treatment parameters may be selected as a type of one or more treatment protocols (treatment protocol may be selected automatically and/or by operator), may be changed automatically by control unit, semi-automatically and/or by operator as described above before and/or during the treatment.

The control unit may communicate with a human-machine interface located on the treatment sleeve and/or on a housing of the device. This human-machine interface may also be part of an external device such as a personal computer, smart phone, tablet and/or other device that may control and/or guide treatment of the patient.

The present device may connect to a mother case as described in U.S. Provisional Application No. 62/375,796 incorporated herein by reference and may communicate and/or cooperate with one or more other devices. In another embodiment cooperation of the present device with other one or more devices may be guided by a remote control that can change at least one parameter of provided treatment. Connection and/or communication with such an external device may be via wire or optical cable, by radio frequency, by infrared and/or other similar wireless means of communication.

The present device may include hardware and/or software in order to provide modularity. The present device may be able to recognize and/or communicate with new treatment pieces, massage/treatment units and/or other treatment devices.

Focusing of a treatment energy source may be provided by placement geometry of an individual active energy source, by design of individual energy source (e.g. parabolic shape of transducer), by superposition of provided electromagnetic field in the treated area, by specific physical properties of treated target area and parameters of provided treatment energy source (e.g. different dielectric properties of fat tissue beside muscle tissue), by phase shift between several treatment energy signals, by wave guides and/or by other mechanism.

The control unit may cooperate with one or more sensors. Information from the at least one sensor may provide information about the treatment parameters and composition of the soft tissue, progress of the treatment and/or other information. The information may be displayed in a human-machine interface.

Feedback information may be e.g. pressure gradients, temperature gradients, which treatment energy sources are activated and inform about parameters of provided treatment energies and/or other information may be displayed on the patient's body, user interface display and/or on the treatment sleeve surface. Such information may be displayed as a colored area with different color intensity, shades of the color and/or by different color across the treated surface. In FIG. 5, 18 is a treatment sleeve with at least part its surface having a display, 19 is a displayed temperature gradient, 20 is pressure gradient and 21 is a displayed active unit that provides some of mentioned above therapy e.g. RF therapy. Displayed information is intuitive distinguished by color and shades. Information may be displayed on a flexible large display on the treatment sleeve surface and/or projected by external projector and/or by projector included in the device. Displays using a special optical layer, optical fibers, waveguide, thermo-active materials and/or by led diodes implemented on the treatment sleeve surface may optionally be used.

Information from the at least one sensor as feedback information may be evaluated by a control unit and/or information may be evaluated by user. Feedback information may be used for switching on/off of one or more treatment energy source and/or in order to modify treatment parameters or protocols to improve effectiveness of the treatment, safety of the treatment, determine soft tissue structure of the treated soft tissue, reduce time of the treatment be more effective of using energy from the power source, or prevent of device malfunction.

Feedback information may be may be measured as received values from specific treatment energy sources, collected by different types of sensors, and may have different characters e.g.: biological, chemical, physical (e.g. an electrochemical sensor; a biosensor; a biochemical sensor; a temperature sensor; an infrared sensor; pressure sensor; a sorption sensor; a sensor measuring absorption and/or backscattering of specific electromagnetic wavelength, a pH sensor; a detector of moving velocity; a gyroscope; a photo sensor; sensor measuring fluid viscosity; a sensor for measuring flow of the liquid/air; a camera; a pressure sensor; a sensor for measuring fluorescence of the patient surface; a sound detector; sensor for measuring of specific heat capacity of a patient soft tissue; a sensor for measuring value of magnetic field; sensor for measuring impedance; sensor for measuring voltage, electrical current; permittivity; conductivity; electric and/or magnetic susceptibility, any suitable sensor or more sensors measuring biological parameters and/or combination thereof e.g.: sensor for measuring dermal tensile forces; sensor for measuring the activity of the muscle; a sensor for measuring muscle contraction forces; sensor for measuring pulse of the patient and/or patient blood pressure; a sensor measuring skin elasticity. The device may also include at least one contact sensor for monitoring applicator contact with body surface of the patient.

Some of the sensors named above may be used for quantification of blood and/or lymph flow (e.g.: sensor measuring backscattering and/or absorption of specific electromagnetic wavelength, ultrasensitive sound detectors, sensor measuring dermal tensile forces, sensor measuring dielectric tissue parameters fluctuation and/or other sensor).

One or more sensors may be used as an imaging system for monitoring structure of the soft tissue. Signals processed for imaging of the soft tissue structure may be part of the treatment energy source delivered to the soft tissue in order to provide treatment of the patient (e.g. backscattered part of the provided energy) and/or imaging signal may be produced by special units relevant one or more types of above mentioned sources. This imaging system may provide measurement and/or may monitor process of the treatment before, after and/or during the treatment and may cooperate with the control unit.

Information from one or more sensors may be displayed. Some of displayed information may be summary composition of the feedback information from one, two or more sensors. For example blood and/or lymph flow may be measured by fluctuation of light absorption and/or fluctuation of backscattered light intensity and wavelengths (e.g. green light color and/or other light wave lengths), by measuring pressure fluctuation in the tissue (veins), and/or by measuring liquid flow based on Doppler's effect from one, two or more different angles with use of acoustic waves and/or other type of waves including ultrasound waves, measuring sounds produced by body liquid flowing. In some embodiment body liquid flow (e.g. blood flow) may also be based on measuring of electromagnetic induction and/or dielectric spectroscopy of soft tissue volume. This may be provided by measuring fluctuation of an applied magnetic field (and or electrical current, electromagnetic field and/or electric field of defined value) and/or induced electrical current in part of the treatment sleeve (e.g. a specialized detector, feedback from an applied source, or a sensor). Fluctuation of such measured and/or indirectly valuated physical quantity are connected to specific ions, molecules and/or elements contained in the body liquid that may change dielectric properties of tested soft tissue volume and/or their movement may induce electrical current under certain condition (e.g. applied magnetic field). These methods of measuring flow of body liquids (e.g.: lymph and/or blood) may include detecting type of body liquid flow (e.g.: laminar, turbulent and/or level of turbulent flow), type of body liquid, direction of flow, temperature of such body liquid, or location and depth in the patient soft tissue of such body liquid. Such parameters may be useful for example during collecting of feedback information that may be used in order to adjust treatment parameters e.g. applied pressure to the patient soft tissue.

The treatment sleeve may or may not encircle a whole body part. The treatment sleeve may be extended by one or more treatment pieces to increase the sleeve size and/or functionality. Treatment pieces may be combined with the treatment sleeve and may provide treatment of any body part and/or any body size with regard to special needs of an individual patient. Treatment pieces of the treatment sleeve may have different properties and/or may provide size extension of the treatment sleeve, may add one or more sensors, may add at least one treatment energy source that provides one or more treatment therapies and/or treatment sleeve parts that may provide better contact with the patient body and treatment sleeve. Another treatment piece of the treatment sleeve may contribute to more hygienic treatment, contribute to more comfortable treatment, safer treatment, treatment energy transfer to the patient's soft tissue and/or may modify treatment parameters (e.g. for individual needs of the patient). Some of the treatment pieces may be disposable and some of them may be used repeatedly.

The treatment pieces of the treatment sleeve described above may have several embodiments. Some treatment pieces of the treatment sleeve may have different mechanical, physical and/or chemical properties (e.g.: filtering of delivered specific treatment energy source signal, influence parameters and/or focusing of such delivered treatment energy source signal and/or other properties).

Some of the treatment pieces may include substances used as heating/cooling medium (e.g.: deionized water, water, ethylene glycol, diethyl glycol, propylene glycol, ethanol, mineral oils, silicone oils, transformed oils, several liquid gasses, gaseous gasses, gels and/or others). The substances may also change parameters of delivered treatment therapy and/or include drugs released to the patient body, as described in U.S. Provisional Application No. 62/331,060 incorporated herein by reference. One or more substances included in the treatment piece may improve treatment results, make treatment more comfortable (e.g. minimize pain during the treatment) and/or safer.

Some of the treatment pieces may include a control unit that may be able to communicate with the control unit of the treatment sleeve and/or any other control unit. Some of the treatment pieces may be connected with the control unit of the treatment sleeve and/or any other control unit and may not include its own control unit.

The released substances may include oleoresin capsicum (pelegrin-acid-vanillilamid) increase the skin blood flow. Other substances may include lipolysis augmentation agents like: methyl xanthines such as theophylline, aminophylline, caffeine; pentoxifylline; beta-1 adrenergic agonists (stimulators) including, but not limited to: forskolin, norepinephrine, epinephrine, isoproteranol; specific beta-3 agonists (stimulators) including but limited to: fenoterol, clenbuterol; alpha-2 adrenergic inhibitors (antagonists) including but not limited to: yohimbine, rauwolscine, oxymetazoline, piperoxane, phentolamine, dihydroergotamine, idazoxin; adenosine inhibitors; calmodulin agonists; thyroid hormones including but limited to: T3/triiodothyronine and T4/tetraiodothyronine; sex hormones including but not limited to methyltestosterone; prostaglandin inhibitors including but not limited to aspirin (ASA), non-steroidal anti-inflammatory drugs (NSAID's), and finasteride, tamoxifen. Named substances are not entire list of possible used substances and other substances may be used to provide the effect described above.

The removable treatment pieces of the treatment sleeve may have different sizes and shapes, which may be adapted to specific anatomical body area. The treatment pieces may be made from at least one material e.g.: ceramic, rubber, polymeric material, metal and/or other.

These treatment pieces may be attached to patient body, attached to treatment sleeve from the inner and/or outer side of the treatment sleeve and/or may be at least partly inserted between layers of the treatment sleeve. Attaching systems of one or more treatment pieces are described in U.S. Provisional Application No. 62/375,796 incorporated herein by reference.

Some embodiments of the treatment sleeve may be applied and adapted to be used on different body sizes (e.g. slim, oversize people etc.) and/or different body portions (e.g. leg, arm, buttock, belly, etc.).

Placement and functionality of the treatment sleeve may be displayed on the human-machine interface.

Functionality of the treatment sleeve and/or its correct fitting may be verified by evaluation of specific feedback information and/or other information from the sensors incorporated in the treatment sleeve and/or connected to the device. Functionality of treatment energy sources, treatment parameters and/or other component of the treatment sleeve may be tested before the treatment, during the treatment and/or also after the treatment. Functionality of treatment sleeve, it's individual parts, one or more connected parts and/or devices may be measured directly by sensor measuring relevant characteristic (e.g. for RF treatment energy source in multipolar embodiment it's functioning may be measured by measuring of current density between electrodes) and/or by indirect measuring by analysis of data from one or more sensors and/or physical variable (e.g. time), that may be influenced by malfunction of specific part of the treatment sleeve and/or malfunctioning of specific treatment therapy (e.g. measuring of infrared part of the spectrum radiated from the patient during the use of RF treatment energy source in order to investigate surface temperature and focusing and/or functioning of RF therapy treatment source).

Another example of indirect detection and testing the treatment sleeve may be a function test of pressure cell and/or correct placement of the treatment sleeve. When the treatment sleeve is properly placed on the limb, pressure cells have a different inflation profile (e.g. characteristic of pressure rising/decreasing and/or reaching of defined pressure in the precise pressure cell during the measured time where inlet air/liquid flow is defined) compared to when treatment sleeve is not in the right place, is not applied in the right way and/or the pressure cell has a defect.

Test of treatment sleeve functionality may also be provided in different ways.

For lymph stimulation is helpful to distinguish anatomical parts and structure of the soft tissue. This may be provided by design of the treatment sleeve, by correct placing of the treatment sleeve. Correct placement of the treatment sleeve may be helped by the system based on structure, anatomical design of treatment sleeve parts and/or software that cooperates with one or more sensors described above, or an imaging device/system. Distinguishing anatomical parts may be important in order to provide effective treatment (e.g. to provide ideal transfer of energies provided by specific treatment therapies, collecting undistorted feedback information, etc.) and to determine treatment protocol and/or its parameters. In some preferred embodiments and/or protocols it may be desirable to stimulate lymph nodes in order to improve flowing of the lymph and its drainage.

The control unit may monitor treatment parameters that may include controlling of massage and/or other treatment units unit(s) that operate in a specific area of the patient body (e.g. by regulation of one or more inflation profile of the pressure cell 1, by regulation of one or more massage units, by adjusting other part of the treatment sleeve and/or its other treatment parameters).

In some embodiments the treatment sleeve may include a monitoring system for monitoring applied pressure. This monitoring system may cooperate with a control unit and may improve: contact with the patient, lymph drainage, massage of the soft tissue and/or other treatment features. Based on this information treatment protocol may be changed or adjusted and/or function test of the treatment sleeve and/or determining correct placement of the treatment sleeve may be performed.

Each treatment unit may have individual control unit and/or may be controlled by a control unit. The control units may be localized in a mother case, in the treatment sleeve and/or may be guided by an external treatment device (e.g.: a smart phone, computer, tablet and/or other devices) as described above. A treatment sleeve may include one or more massage/treatment units.

In the device of FIG. 1, the massage unit includes element changing pressure value on the patient's soft tissue. Element changing pressure value on the patient's soft tissue is any treatment energy source providing massaging effect without significantly damaging cells in the soft tissue and/or without damaging natural soft tissue structure (e.g. may release capillaries by disruption deposits in them but do not damage fat cells, collagen fibers etc.). Massaging effect causes massage of treated soft tissue. Element changing pressure value on the patient's soft tissue may be any treatment energy source, described in this document, providing energy on the patient soft tissue with parameters that cause massaging effect.

Element changing pressure value on the patient's soft tissue may be included in the treatment sleeve (e.g. between any layers of the treatment sleeve) and/or out of the treatment sleeve (e.g. to the patient body and/or located externally where distance between element changing pressure value and patient's surface is longer than distance between any treatment sleeve layer and patient's surface).

In some embodiments a massage unit may include more elements changing pressure value e.g. may be divided into several pressure cells 1, as shown in FIG. 2. The pressure cells may be filled with liquid and/or gas (e.g.: air, CO2, N2 and/or other gas). The massage unit may alternatively comprise massage objects as described below.

Pressure value applied on the patient's soft tissue means that a treatment energy source providing massaging effect applies positive, negative and/or sequentially changing positive and negative pressure on the treated and/or adjoining patient's soft tissue structures and/or creates a pressure gradient under the patient's soft tissue surface. Some massage units may have an element changing pressure value causing massaging effect by different ways other than by creating a pressure gradient on the soft tissue. Massaging effect may also be caused by creating thermal gradient in the lymph, muscle stimulation, influencing vasoconstriction, vasodilatation, permeability of capillaries and/or other mechanism). Treatment protocol may influence the layer of soft tissue structure (e.g. muscle, dermis, hypodermis and/or other) to be treated.

Massage may be provided with and/or without contact with the patient's soft tissue, depending on the treatment energy source providing massage. The element changing pressure value on the patient's body may be an acoustic wave or air nozzle in order to create pressure gradient in the patient's soft tissue without contact the patient's soft tissue.

Negative pressure is pressure below atmospheric pressure. Positive pressure is pressure value above atmospheric pressure. Atmospheric pressure is pressure of the air in the room during the therapy.

Massage applied in order to improve body liquid flow (e.g. lymph drainage) and/or relax tissue in the surface soft tissue layers may be applied with pressure lower than during the massage of deeper soft tissue layers. Such positive or negative pressure may be in range of 10 Pa-30 000 Pa, more preferably 100 Pa-20 000 Pa even more preferably 0.5 kPa-19 kPa or the most preferred range may be 1 kPa-15 kPa.

Massage applied in order to improve body liquid flow and/or relaxation of the tissue in the deeper soft tissue layers may be applied with higher pressure. Such positive or negative pressure may be in the range from 10 kPa to maximal pain threshold of the patient more preferably in range from 12 kPa to 400 kPa or even more preferably from 15 kPa to 300 kPa or the most preferably from 20 kPa to 200 kPa. An uncomfortable feeling of too high applied pressure may be used to set a pressure threshold according to individual patient feedback.

Negative pressure may stimulate body liquid flow and/or relaxation of the deep soft tissue layers (0.5 cm to non-limited depth in the soft tissue) and/or layers of the soft tissue near the patient surface (0.1 mm to 0.5 cm). In order to increase effectiveness of the massage negative pressure treatment may be used followed by positive pressure treatment.

Cycles of the massage therapy (repeated pulse sequences, treatment patterns and/or repeated parts of the treatment protocols) and its duration may be variable depending on values of applied pressure, methods of applied pressure and/or type of the treatment sleeve used.

Pressure applied by a static element changing pressure to one area of the soft tissue without changing position of the massage unit may be variable in range from 1 ms to 45 minutes more preferably 0.5 s to 10 min, even more preferably 1 s to 5 min or the most preferably 2 s to 30 s. Values of applied pressure may be variable during the cycle of the massage.

Pressure applied by movable element changing pressure on the soft tissue may have no time limitation of use or in some special treatment protocol may be in range between 30 seconds to 10 minutes or between 30 s to 5 minutes or between 30 s to 3 minutes.

A number of elements changing pressure values on the patient's soft tissue may be in treatment between 1 to 6000 element changing pressure values (e.g. pressure cells) or more preferably between 8 to 80 element changing pressure value (e.g. pressure cells) or even more preferably between 2 to 40 element changing pressure value (e.g. pressure cells) or most preferably between 4 to 32 element changing pressure value (e.g. pressure cells) may be used.

Sizes and/or shapes of elements changing pressure value to the patient's soft tissue (e.g. pressure cells) may be different according to treated area. One element changing pressure value on the patient's soft tissue (e.g. pressure cells) may cover an area on the patient surface between 1 cm² to 1 m² or more preferably between 10 cm² to 1000 cm² or even more preferably between 40 cm² to 800 cm² or the most preferably between 150 cm² to 600 cm².

Several pressure cells and/or other elements changing pressure value on the patient's soft tissue may work simultaneously and/or switching between them may be in intervals between 5 ms to 10 s or more preferably 0.1 s to 5 s or the most preferably interval between 0.5 s to 2 s.

A element changing pressure value in order to provide massaging effect may be guided according to one or more predetermined massage profile included in the one or more treatment protocols. The massage profile may be selected by the operator and/or by a control unit with regard to the patient's condition. For example a patient with lymphodema may require a different level of compression profile and applied pressure than a patient with a healed leg ulcer.

The following examples show several operating programs of massage units:

| | Recommended | | |
|---|---|---|---|
| | pressure range | Recommended | Characteristic and effects of the |
| Name | [kPa] | time [min] | program |
| Massage | 5-11 | 30 | The chambers are inflated and |
| | | | deflated in succession. The effect is |
| | | | similar to that of a superficial |
| | | | massage. |
| Physiological | 3.5-9.5 | 30 | Removes stasis and encourages |
| | | | rehabilitation of the vascular |
| | | | system. Mainly used for |
| | | | regeneration and sport medicine. |
| Preparation | 3.5-9.5 | 20 | Preparative program to be used to |
| | | | stimulate the body's tissues before |
| | | | further lymphatic treatment. |
| Lymph | 3.5-9.5 | 45 | Similar to manual lymphatic |
| drainage | | | drainage massage. Starts by |
| | | | unblocking the groin or axillar |
| | | | ganglion. This is the most suitable |
| | | | program for aesthetic medicine. |
| Elephantiasis | 3.5-11 | 45 | The chambers are inflated in |
| | | | succession and then remain |
| | | | inflated. This program is suitable for |
| | | | marked lymphostasis and |
| | | | significant retention of lymphatic |
| | | | fluid. |
| Venopress | 2.5-7 | 30 | The program for support and |
| | | | increase of blood flow in |
| | | | peripheries. Helps to prevent |
| | | | vascular problems like varices or |
| | | | phlebedemas. |
| Embrocation | 3.5-9.5 | 45 | Sequential inflating of single |
| | | | chambers in order to ensure careful |
| | | | removing of lymphatic fluid. |
| Reversed | 3.5-9.5 | 45 | Combined program where |
| combi | | | chambers are inflated in preset |
| | | | pattern. Starts by releasing the |
| | | | stasis, continues by successively |
| | | | pushing the lymphatic mass |
| | | | proximally. |

One or more elements changing pressure value and/or other part of the treatment sleeve may provide constant pressure along the whole treatment sleeve during the treatment. This pressure may be variable during the time of the therapy but pressure applied to the patient body may still be the same across every treated body location.

In another treatment protocol and/or other device embodiment, pressure applied by specified massage unit(s) to the patient body may be constant during the treatment but may be different at different treated areas and/or a pressure value ratio between at least two locations may be constant e.g. pressure to calf muscle may be lower than pressure applied to area around the knee. When the applied pressure to the calf is changed, the pressure to the knee area is changed with the same ratio.

According to another embodiment and/or treatment protocol pressure applied to the patient body may be different in different soft tissue areas and each element changing pressure value and/or group of them may be individually guided and produce different and variable pressure on the patient soft tissue during the treatment.

Massage and or other therapy may be provided in a pattern that is created by switching between previous and successive treatment energy sources. For example, in FIG. 2 a massage unit with pressure cells may create pressure individually, simultaneously, sequentially and/or with overlay in cooperation with one or more other elements changing pressure value. Treatment protocol may include information about a massage/treatment pattern e.g. which, when and/or for how long massage unit(s)/treatment unit(s) are switched on/off. Other characteristics of the treatment protocol for a defined massage/treatment pattern are parameters of delivered treatment energy source(s) and/or pressure parameters of the massage unit or group of massage units that are activated (switched on/off) according defined contiguity. A treatment pattern may simulate movements with an activated treatment energy source. FIG. 7 shows various treatment patterns. Treatment patterns may simulate: linear moves that may overlap e.g. as shown in FIG. 7 A)-C); curvilinear moves e.g. as shown in FIG. 7 D); curvilinear moves that may imitate progressive circular moves e.g. as shown in FIG. 7 E)-I) and/or a combination of these as shown in FIG. 7 J)-L).

Massage/treatment patterns in some treatment protocols may simulate massage/treatment provided by a physiotherapist and may enhance massage/treatment provided by a physiotherapist. The present apparatus and methods are not limited to just the two hands of a physiotherapist, or by the strength and fatigue of a physiotherapist. Massage provided by the present designs may also be provided in a different manner than applied mechanical pressure e.g. acoustic waves that may be more targeted and/or may selectively provide massage of deep soft tissue layers without influencing upper soft tissue layers that a physiotherapist is not able to provide. This massage/treatment pattern may change during the treatment.

Pressure applied by one or more massage units may be gradually applied preferably in the positive direction of the lymph flow and/or the blood flow in the veins. According to specific treatment protocols the pressure may be gradually applied in a direction opposite or different from ordinary lymph flow. Values of applied pressure during the treatment may be varied according the treatment protocol.

A pressure gradient arises between individual massage units and/or between elements changing pressure value to the patient's soft tissue included in one massage unit. A massage unit may have more than two elements changing pressure value (e.g. more than two pressure cells). Examples of gradients described are not limited for this method and/or device. The setting of the pressure gradient between at least two previous and successive treatment energy sources and equivalently between previous and successive elements changing pressure may be: 0%, i.e. The applied pressure by massage units is the same (e.g. pressure in all chambers of the treatment sleeve is the same); 1%, i.e. The applied pressure between a previous and a successive elements changing pressure value decreases and/or increases with a gradient of 1% (e.g. the pressure in the first chamber is 5 kPa and the pressure in the successive massage is 4.95 kPa); 2%, i.e. The pressure decreases or increases with a gradient of 2%. The pressure gradient between two elements changing pressure value to the patient's soft tissue may be in range 0% to 100% where 100% means that one of elements changing pressure value is not active and/or does not apply any pressure on the patient's soft tissue. In a preferred embodiment and/or treatment protocol the applied pressure gradient between a previous and a successive treatment energy sources represented in massage therapy by elements changing pressure value may be in range between 0% to 95%, or in even more preferred embodiment and/or treatment protocol applied pressure gradient between a previous and a successive elements changing pressure value may be in range between 0% to 70%, or in the most preferred embodiment and/or treatment protocol applied pressure gradient between a previous and a successive elements changing pressure value may be in range between 0% to 50%.

Various treatment patterns and pressure gradients may be achieved by treatment sleeve comprising plurality of treatment energy sources and/or elements changing pressure value arranged in e.g. line or matrix by providing successive therapy. A successive and a previous treatment energy sources may be any kind of the treatment energy sources. The successive and the previous treatment energy sources and/or equivalently successive and previous elements changing pressure value have to comply with the prescribed requirements:
The successive and the previous treatment energy sources provide variably output power during the treatment.
The successive treatment energy source provides the same treatment therapy as the previous treatment energy source.
The successive treatment energy source is included in the same treatment sleeve as the previous treatment energy source.
The successive treatment energy source is the source which starts to increase the output power value after the previous treatment energy source increases its output power value.

According to one embodiment more than one successive treatment energy sources may increase output power at the same time.

It is possible to designate more than one treatment energy sources as the previous treatment energy source that is distinguished from the previous treatment energy source for specific one successive treatment energy source and it is the one with the shortest distance from the successive treatment energy source.

Designation of a previous and successive treatment energy source is changing during switching on/off or changing output power between individual treatment energy sources in order to simulate movement of the treatment energy during the therapy.

When a successive treatment energy source activated, a previous treatment energy source may or may not be still activated.

A treatment energy source is considered as activated when it is providing any wanted treatment energy type (any treatment therapy) to the patient's soft tissue.

Successive therapy creates a pattern and may be defined relative to successive therapy speed. The successive therapy speed describes speed of moving changed intensity of treatment energy source/elements changing pressure value along the treatment pattern. Successive treatment therapy may be provided by switching between previous and successive treatment energy sources and/or changing output power between a previous and a successive treatment energy sources. The successive therapy speed is then counted as a division of distance between central parts of a previous and a successive treatment energy sources and time delay between starts of output energy rising of a previous and a successive treatment energy sources. The successive therapy may be also be provided by moving of the treatment energy source relative to the patient's surface. Then the successive therapy speed is moving speed of the treatment energy source. According to still another embodiment the successive therapy may be achieved by changing spatial coordinates of a focal spot across the treated body area (changing of focus depth in the soft tissue is not included). Then successive therapy speed is speed of moving center of focal spot across the patient soft tissue. The successive therapy speed is measured in units of cm·s⁻¹.

Average successive therapy speed may be in range between 0.1 cm·s⁻¹ and 50 cm·s⁻¹ or more preferably in range between 1 cm·s⁻¹ to 30 cm·s⁻¹ or the most preferably in range between 5 cm·s⁻¹ to 15 cm·s⁻¹.

Rates of pressure change applied on the patient's soft tissue (e.g. by inflation of the pressure cells) may be a time and pressure function described by a constant function, linear function, exponential function, logarithmic function, sinus function and/or combination of this functions in the meaning of superposition of two or more described functions in the same and/or different time domain.

According to some embodiments at least some of the elements changing pressure value may be interconnected and air and/or liquid may flow across one or more massage units. In some embodiments massage units may be separated from each other.

A massage unit may include one or more movable parts (elements changing pressure value), inflated/deflated by air and/or liquid, which may change volume and/or shape in order to create pressure to the patient body (e.g. may be created from thermal, magnetic and/or electrical shape changing materials). Shape and/or volume changing materials may be alloys e.g.: NiTi, NiTiCu, CuZn, CuZnAI, CuAlNi, FeNiAl, FeMnSi and/or ZnAuCu. In some embodiment the massage unit may be based on massage objects (elements changing pressure value) pushed against the patient body by electrostatic, electromagnetic, magnetic, mechanical force (e.g.: caused by air and/or liquid flow/expansion) and/or combination of thereof.

FIG. 4 illustrates a cross section of an embodiment (e.g. a treatment sleeve) with multiple layers 14-17 placed on the patient body 13.

First layer 14 of the treatment sleeve may be an air gap and/or may be filled with one or more substances that may accelerate or improve treatment results, to make treatment more comfortable, may accelerate treatment and/or make the treatment safer with minimal side effects. Examples of such substances are described in U.S. Provisional Application No. 62/331,060 incorporated herein by reference. In some embodiment first layer 14 may not be included in the treatment sleeve. A protecting layer 15 may be located above the first layer 14. The protecting layer 15 may be made from different materials (textile, polymeric, metal, ceramic, rubber and/or other material). This protecting layer 15 may have different physical and chemical properties. Protecting layer 15 may be breathable or non-breathable, antibacterial, conductive, nonconductive, hydrophilic and/or hydrophobic. Multiple protecting layers may be used. One or more of the protecting layers may be removable. The protecting layer 15 may have a hydrophilic side that helps remove sweat from the patient body. The protecting layer 15 may have a hydrophobic side that prevents contact between cooling fluid and/or other fluid contained in the treatment sleeve with the patient. One side of protecting layer may be hydrophilic and other side may be hydrophobic to allow the protecting layer to perform a specific function. Layer 16a represents layer having at least one treatment unit 16b with at least treatment energy source. Treatment unit 16b may provide a different type of treatment energy source (e.g. for massage and/or other treatment) or a sensor. Layer 17 in FIG. 4 is an outer protecting layer of the treatment sleeve. This layer may also include a cooling system.

FIG. 4 shows one example of a treatment sleeve. Sensors, treatment energy sources, protecting layers and/or other parts of the treatment sleeve may be arranged in different ways and different numbers of layers may be used.

A massage unit may have a changeable surface curvature where curvature of the surface may be changed at least across part of such massage unit. This effect may be achieved e.g. by a roller and/or movable element changing pressure value called also massage object located behind a protecting layer and/or in contact with the patient; by a shape changing material (e.g. electrical shape changing material implemented in such massage unit may change the surface geometrical conformation); by inflating/deflating an internal cavity of the treatment sleeve; by mechanical forces and/or other mechanism changing the volume and/or shape of space inside the treatment sleeve.

In another embodiment the massage unit may be designed with a treatment sleeve with a flat or bulging surface and pressure may be applied by shrinking part of the treatment sleeve and/or by moving one or more parts.

Parameters of size and shape of the massage unit and element changing pressure value (e.g. pressure cell) are not limited and may be the same or different across the treatment sleeve. The shape of the massage unit may be symmetrical and or asymmetrical in order to comply with the surface of the patient.

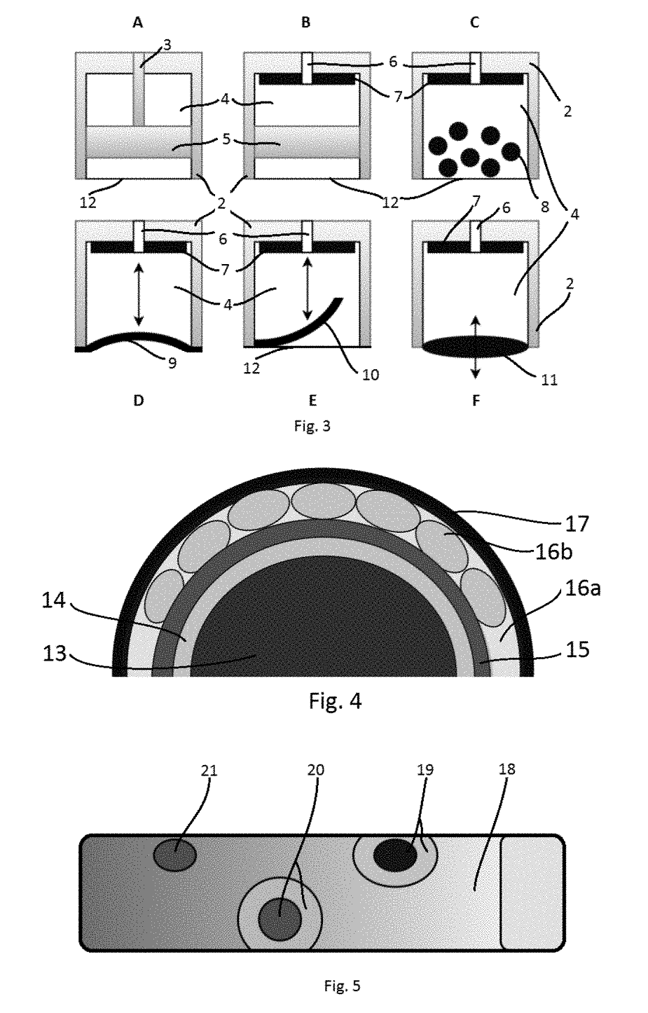

Increasing/decreasing pressure applied to the patient soft tissue may be accompanied with vibration. Vibration may be created by sequential or random inflating/deflating; by moving particles in the pressure cell/gap volume 4 shown in FIG. 3; by rotation of an object and/or by other source of vibration, e.g. mechanical waves; magnetic field, electric field, electromagnetic field and/or electrical charge, in order to accelerate at least one massage object and/or to accelerate at least part of one or more massage objects (such as a vibrating membrane 9 as shown in FIG. 3) in at least one massage unit. Energy that accelerates a massage object may also be provided from air expansion and/or flow of air or liquid. Air expansion and/or flow of air or liquid may be combined with other types of energies in order to provide more effective massage of soft tissue. Flow of liquid and/or air into the massage unit where the massage object may be located and/or into the piston and/or chamber connected to massage object may also regulate the massaging effect of the massage unit.

Kinetic energy of accelerated one or more massage objects may be used for massage of the patient's soft tissue and/or in order to create pressure to the patient soft tissue. These massage objects may be incorporated under the pressure cell 1 shown in FIG. 2 and/or in the pressure cells 1, in other massage units shown in FIG. 3 and/or in different layers of the treatment sleeve (e.g. in contact with patient surface and/or massage unit may be covered by a protecting layer).

Number, size, weight, material and/or shape of the accelerated massage object or objects may be variable. Massage units may also have different sizes, shapes, mechanisms of providing massage/pressure to the patient's body and/or may be made from different types of at least one material.

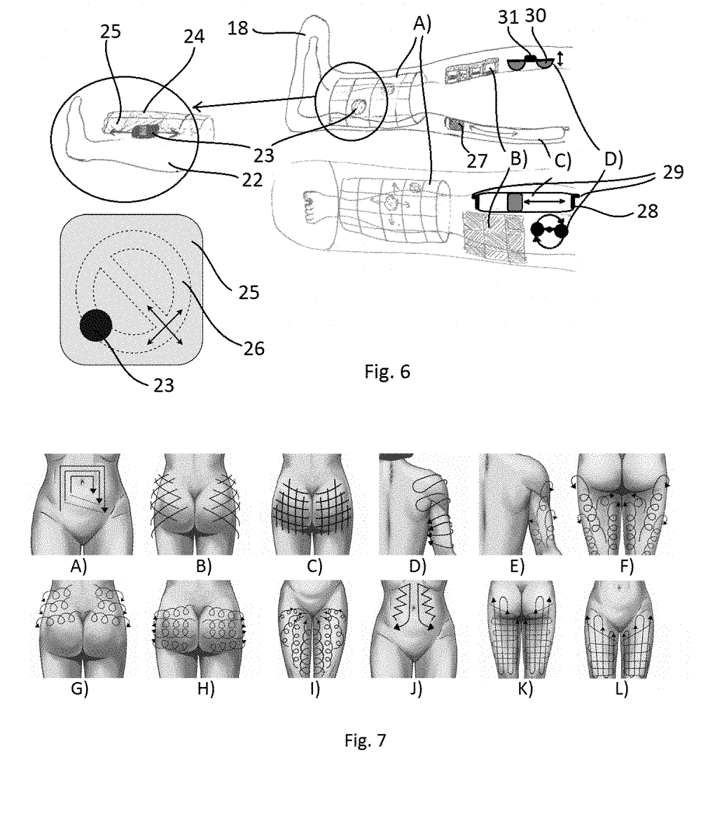

One type of massage unit has a static element changing pressure value on the patient's soft tissue and the other type is with movable element changing pressure value on the patient's soft tissue. The static type of element changing pressure value is not able to change position of target area (focus spot side and focus depth not included) and/or location in the treatment sleeve, except via the operator. The movable element changing pressure value is able to change a targeted area of applied pressure and/or is able to change location in the treatment sleeve during the treatment (e.g. at element changing pressure value may move across the treatment sleeve). Additionally one or more massage units and/or elements changing pressure value may be movable and may continually move across the patient, as shown in FIG. 6.

FIGS. 6 A, C, and D illustrates three embodiments of movable treatment energy sources (e.g. elements changing pressure value) and one complex of static massage units FIG. 6 B. In FIG. 6,18 is the treatment sleeve, and 22 is the leg of the patient.

Several examples of static massage units are illustrated as a complex of massage units B) in FIG. 6. Detailed description of individual massage unit is illustrated in FIG. 3 unit A-F.

FIGS. 3A-3F shows several different massage units embodiments. FIG. 3A illustrates an impacting massage object 5 powered by a piston 3. Massage object 5 is located in a chamber 2 of the massage unit. A gap volume 4 between massage object 5 and chamber 2 may be filled by air, liquid or a vacuum during the moving of the massage object 5. Massage object 5 and/or whole massage unit may be separated from the patient body by a protecting layer 12.

In FIG. 3B, the massage object 5 operates by filling and/or sucking out liquid and/or air from the gap volume 4 by an inlet/outlet valve 6. The valve 6 may alternatively be separate from the massage unit. Massage object 5 may also be powered by an element 7 that creates an electric field, magnetic field and/or electromagnetic field. The element 7 may also provide electric, magnetic electromagnetic field or therapy.

In FIG. 3C massage may be provided by impacting of multiple massage objects 8. The multiple massage objects 8 may have the same or different size, shape, weight and/or may be created from the same or different materials. The massage objects 8 may be accelerated by air or liquid flowing (through the valve 6) and/or by an electric, magnetic and/or electromagnetic field (e.g. by the element 7). Trajectory of the massage objects may be random, circular, linear and/or massage objects 8 may rotate around one or more axes, and/or may do other types of moves in the gap volume 4.

In FIG. 3D, a membrane 9 may be accelerated by an electric, magnetic, electromagnetic field (e.g. by the element 7) and/or by changing pressure value in the gap volume 4 between wall of the chamber 2 and membrane 9. A piston and/or other object may be directly connected to membrane 9 to change curvature of the membrane 9. Membrane 9 may also be replaced by a protecting layer 12, as shown in FIG. 3E.

The membrane 9 may be made from thermal, magnetic, electrical shape changing materials from at least partially elastic, polymeric, metal or other material. Charge that may cause moving of the membrane maybe brought directly to the membrane 9 and/or to the wall of the chamber 2. Magnetic field and/or electromagnetic field may be generated by element 7. Membrane 9 may not cover the whole bottom of the massage unit. The protecting layer 12, if used, may substitute for the membrane 9 and provide the function of the membrane 9.

In FIG. 3E the same principle described above for acceleration of the massage object 10 is used with the protecting layer 12 that has at least one edge free to oscillate.

In FIG. 3F a massaging object 11 has thermal, magnetic and/or electrical shape changing materials that is able to change volume and/or curvature under the influence of temperature, magnetic, electric field and/or distributed electrical charge in volume and/or surface of the massaging surface 11.

Massaging surface 11 embodiment may be made from at least partially elastic material and this massaging surface 11 may be repeatedly inflated or deflated by air or liquid and/or may change size, shape and/or curvature under other applied forces e.g. mechanical forces.

FIG. 6 shows an example of a movable treatment energy sources A) having an accelerated treatment energy source e.g. massage object 23 that creates massage effect and/or other treatment therapy by moving treatment energy source across the treatment sleeve 18 along a substrate 24. Substrate 24 may or may not encircle a whole limb and/or body part. Substrate 24 may be divided into several guiding parts 25. Guiding parts 25 may be separately spaced out in the treatment sleeve and/or may be interconnected. The treatment energy source e.g. massage object 23 may be moved in several different ways. The treatment energy source e.g. massage object 23 may be accelerated and moved to specific locations in the substrate 24 by an electric, magnetic and/or electromagnetic field generated by guiding part 25 that is designed from conductive or semi-conductive material.

Another moving mechanism for the treatment energy source e.g. massage object 23 may be provided along rails 26 included in the substrate 24 and/or in the guiding part 25. The treatment energy source e.g. massage object 23 may or may not be in mechanical connection with such rails. The shape of the rails 26 may be different across different guiding parts 25 in order to provide a desired massage pattern. The rails may be interconnected between different guiding parts. Movement of the treatment energy source e.g. massage object 23 may be provided also by electric, magnetic, electro-magnetic and/or mechanical forces. Mechanical forces may be generated by air/liquid flow, an electric motor and/or by any other mechanism. Massage object is a source of energy that provides mechanical pressure on the soft tissue.

Described methods of moving with the massage object 23 may use different types of treatment energy source providing different treatment therapy with different results.

Referring still to FIG. 6, a second example of a movable treatment energy source massage object is treatment unit C) is similar to massage unit B shown in FIG. 3, with difference that treatment e.g. massage provided by treatment unit C) of FIG. 6 may be provided along the whole trajectory of moved treatment energy source e.g. massage object 27. In this example, the massage object may be moved by an electric, magnetic, electromagnetic field generated by element 28, by mechanical forces (similar to element 7 of FIG. 3) and/or by liquid/air flow through the inlet/outlet valve 29 (similar to inlet/outlet valve 6 from FIG. 3). The wall of the massage unit C) of FIG. 6 may be at least partially created from a non-rigid material (e.g. silicone, textile and/or other polymeric materials).

A third embodiment of a movable treatment energy source in treatment unit D) in FIG. 6 may contain at least one treatment energy source e.g. massage object 30, or several massage objects 30 connected and/or attached to a mover 31 (e.g. an electromotor) that accelerates the massage object(s) 30. Treatment unit D) in FIG. 6 may do several types of at least partially circular moves with the treatment energy source e.g. in order to stimulate massage effects. Treatment energy source e.g. massage object 30 may also move in vertical direction to patient's surface.

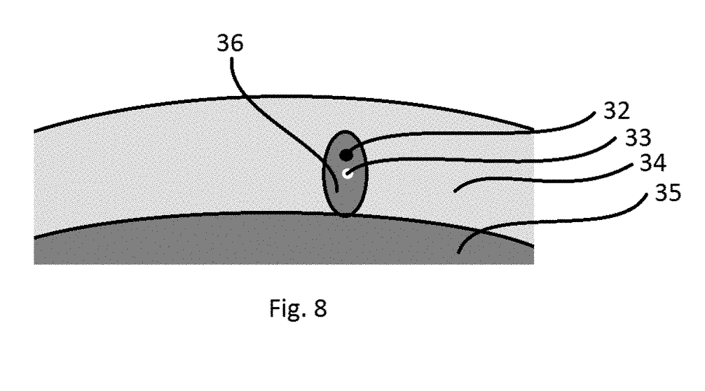

Referring to FIG. 8, a massage object 36 in the treatment sleeve 34 may be symmetrical or asymmetrical. Symmetry of a massage object may be elliptical, circular and/or other type. The massage object 36 may be made from different types of material with different density and/or different mechanical properties. The massage object 36 may rotate around a rotational axis at the center of symmetry 33, out of the center of symmetry, in the center of gravity 32 and/or out of center of gravity. Rotation of such massage object 36 creates massage effect to the patient's surface 35.

In order to improve massage and/or different treatment energy source (e.g.: magnetic field, electric, electromagnetic field, mechanical energy, RF treatment energy source, light energy source, plasma, heating, cooling, acoustic wave treatment energy source, shock wave treatment energy source and/or other type of treatment energy source) may be used. Several treatment energy sources providing different therapy than massage may have synergic effect with provided massage.

In one embodiment acoustic waves may be used in order to provide another massage effect of the surface and/or deeper layers of the soft tissue. This type of massage may be used in continual and/or pulse mode. The acoustic wave may be focused to a specific area under the skin of the patient. Massage by acoustic waves may create areas with higher pressure without influence on other areas of soft tissue. This feature may be used for enhancing lymph drainage, tissue relaxation, metabolism and blood stimulation. Acoustic waves may be targeted and adjusted according to specific needs. Acoustic waves and ultrasound waves treatment energy source may be adjacent to the patient's soft tissue in direct contact with the patient skin (surface) or may be applied without touching the patient surface separated of patient's surface by at least one layer of the treatment sleeve, gel solution and/or air gap (non-contact). Ultrasound waves and/or acoustic waves may be also used for imaging.

Waves used for acoustic massage may be in the range of 0.1 Hz to 20 kHz or more preferably in range 1 Hz to 5 kHz or most preferably range 1 Hz to 1000 Hz. The energy flux density of the acoustic waves is preferably in the range between 0.001 mW·mm⁻² to 160 mW·mm⁻², more preferably in the range between 0.001 mW·mm⁻² to 100 mW·mm⁻², most preferably in the range between 0.01 mW·mm⁻² to 80 mW·mm⁻².

Ultrasound treatment energy source may operate in contact and/or without contact with the patient ‘surface. Adjacent ultrasound treatment energy source to the patient's soft tissue may or may not be divided from the patient surface one or more protecting layers, materials and/or air gap. Ultrasound waves are provided in range from 15 kHz to 4 MHz or more preferably from 20 kHz to 3.5 MHz Energy flux density provided to the patient's soft tissue may be the same as for acoustic waves, with a maximal energy flux density about 20%, 30% or 40% higher than as described for acoustic waves. The minimal energy flux density of ultrasound treatment energy source may be 0.001 mW·mm⁻² or 0.01 mW·mm⁻². The maximal energy flux density of ultrasound treatment energy source may be 224 mW·mm⁻² or 192 mW·mm⁻² or 140 mW·mm⁻² or 112 mW·mm⁻² or 96 mW·mm⁻².

In continual mode acoustic waves and/or ultrasound waves may be applied longer than 20 minutes or more preferably no longer than 20 minutes, even more preferably no longer than 10 minutes or most preferably no longer than 3 minutes. After the end of continual mode a pause at least 1 s is recommended and then acoustic waves may be applied again.

In pulse mode of acoustic waves and/or ultrasound waves one pulse include at least part of one signal period and/or at least part of more than one signal period (signal is measurable value of treatment energy source delivered to the patient's soft tissue). During one pulse signal oscillation creates one continuous function. The signal may last between 0.1 ms to 0.2 s. The most preferred length of one pulse may be between 0.5 ms and 100 ms.

One burst may include at least one pulse. A pause between two pulses in one burst may be variable and may last between 0.1 ms to 0.5 s or more preferably between 1 ms to 200 ms or the most preferably between 1 ms to 100 ms. One burst may last between 2 ms to several minutes (e.g. 40 minutes) or more preferably between 1 ms to 240 s or even more preferably between 1 ms to 100 s or the most preferably between 3 ms to 5 s. A pause between two bursts may be in range from 5 ms to 40 minutes or more preferably in range from 5 ms to 120 s or even more preferably in range from 5 ms to 5 s or the most preferably in range from 5 ms to 2 s.

According to still another embodiment muscle stimulation or other soft tissue structures stimulation by electrical current and/or by magnetic field may be also used as type of massage treatment therapy and/or other treatment therapy than massage. Muscle stimulation may improve targeting of heating up of soft tissue, provide better homogeneity in delivered energy, prevent local hot spots, improve blood and lymph circulation and/or influence dielectric properties of specific soft tissue layers (e.g. may synergistically influence transfer of RF waves into the soft tissue). Repeated muscle contraction accelerates body metabolism, heats up adjoining soft tissues, stimulates secretion of several hormones and/or may be beneficial for body shaping. Muscle contraction causes massage of adjoining soft tissue structure and cause massage of the deep soft tissue layers without affecting the surface of the patient.

Different nerves and soft tissue structures may be stimulated using interferential electrotherapy with a medium frequency in the range of 500 Hz to 12 kHz or in a more preferred embodiment in the range 500 to 8 kHz, in the most preferred embodiment in the range 500 to 6 kHz, creating pulse envelopes with frequencies for stimulation of the nerves and tissues e.g. sympathetic nerves (0.1-5 Hz), parasympathetic nerves (10-150 Hz), motor nerves (10-50 Hz), smooth muscle (0-10 Hz), sensor nerves (90-100 Hz), nociceptive fibers (90-150 Hz).

Muscle stimulation may be provided by e.g. intermittent direct currents, alternating currents (medium-frequency and TENS currents), faradic current as a method for multiple stimulation and/or others. Frequency of the currents and/or its envelope is typically in the range from 0.1 Hz to 200 Hz in preferred embodiment or from 0.1 Hz to 150 Hz in more preferred embodiment or from 0.1 to 140 Hz in the most preferred embodiment.

The method of nerve/muscle stimulation by magnetic field may use a peak to peak magnetic flux density on a coil surface at least 0.2 T, 0.4 T, 1.5 T, 2 T, at least 3 T, or up to 7 T. The repetition rate may be 1 Hz-700 Hz or more preferably 1 Hz-300 Hz or most preferably 1 Hz-200 Hz, with initial or successive treatments lasting several seconds or longer, for example, for at least 5, 10, 30, 60, 120 or 240 seconds, or longer. The pulse width is in the range of tens to hundreds of microseconds.

Stimulation of patient's soft tissue by magnetic field and/or electric field may be used with or without contact of such treatment energy source with the patient's surface.

A treatment energy source may also provide another treatment by a generated magnetic field and/or electric current. The most preferred frequency ranges for individual types of treatment are:
2-10 Hz—endogenous opioid theory—chronic pain management;
60-100 Hz—gate control theory—acute pain management;
120-140 Hz—peripheral pattern theory—subacute pain management;
5 and 150 Hz—fracture healing;
45 Hz—joint mobilization;
2-70 Hz—myostimulation.

Treatment of patient soft tissue may be provided by one or more sources providing electromagnetic field therapy and one or more sources providing massage of the patient soft tissue (e.g. radiofrequency waves). Electromagnetic field therapy may also be combined with other types of the treatment energy sources (e.g., plasma, light, ultrasound, shock waves, acoustic waves and/or with a source of energy that is able to provide heating cooling of the soft tissue). Some of them e.g. plasma and radiofrequency may be targeted and/or influenced by other ways e.g. increased permeability of RF into the soft tissue.

Radiofrequency waves may be delivered to the patient's soft tissue at a specific frequency and/or group of frequencies, of defined intensity and delivered by specific methods (monopolar, unipolar, multipolar method of RF delivered energy) that depends on the type of the stimulated/treated soft tissue layer and/or treatment protocols.

RF treatment may have several benefits for surface micro-circulation of lymph, blood and/or the stimulation of the lymphatic system, which synergistically superpose on the benefits obtainable with endodermic massage to lymph drainage, blood circulation, the re-establishing of skin tone, the cellular exchange and/or improving efficiency of followed treatment therapy.

RF scanning may disrupt ion balance, create a thermal gradient, increase volume of certain layers of the soft tissue, and/or increase fluidity of the body liquids in the treated area by focused RF. This effect, as described above, may significantly affect lymph drainage and other therapies in a positive way. Some types of radiofrequency waves may not influence soft tissue by heating, whereas other types of radiofrequency waves may be used in order to preheat and/or heat soft tissue of the patient.

An electromagnetic field used for heating soft tissue may be typically in the range of 0.1 MHz to 25 GHz. Waves of the RF therapy may be delivered preferably in the range from 90 kHz to 3 500 kHz or 6 765 to 6 795 kHz or 13 553 to 13 567 kHz or 26 957 kHz to 27 283 kHz or 40.66 to 40.7 MHz or 433.05 to 434.79 MHz or 902 to 928 MHz or 2 400 to 2 500 MHz or 5 725 to 5 875 MHz or 24 to 24.25 GHz or 61 to 61.5 GHz or 122 to 123 GHz or 244 GHz to 246 GHz or optionally at other frequencies as well.

In a more preferred embodiment RF waves in the frequency range between 90 kHz to 40.7 MHz are used in order to prevent standing waves in the patient body.

An electromagnetic field may be applied to the patient body in continual and/or pulse modes. Continual irradiation of a body area by RF may be at least 5 s or 20 s or 30 s or 60 s or 120 s or 240 s or 10 minutes or 20 minutes or more than 20 minutes or the most preferably more than 35 minutes.

The pulsed electromagnetic field may last between 50 μs to 100 s, in more preferred protocol pulse may last between 1 s to 70 s, and in the most preferred embodiment pulse may last between 3 s to 70 s.

An RF treatment energy source may be adjacent to the patient's soft tissue in contact mode where RF treatment energy source (electrode) is in contact with the patient's surface, indirect and/or in no-contact mode RF treatment energy source is not in contact with the patient's surface. In noncontact mode RF treatment energy source may be provide from externally of the treatment sleeve and/or may be in the treatment sleeve spaced from the patient's surface by conductive, nonconductive layer, air gap, gel solution, water bolus and/or other at least one layer.

Energy flux density (energy flux density on the electrode surface) of the electromagnetic field in noncontact mode, where electrodes providing RF signal are spaced from the patient body by a material of low relative permittivity near to relative permittivity of the air and/or by a spacing object with non-beneficial dielectric properties, may be preferably in the range between 0.01 mW·mm⁻² and 10 W·mm⁻², more preferably in the range between 0.01 mW·mm⁻² and 1 W·mm⁻², most preferably in the range between 0.01 mW·mm⁻² and 400 mW·mm⁻².

Energy flux density of the electromagnetic field in contact mode (including the direct contact of electrodes coated by thin layer of insulator) may be preferably in the range between 0.01 mW·mm⁻² and 2 000 mW·mm⁻², more preferably in the range between 0.01 mW·mm⁻² and 500 mW·mm⁻², most preferably in the range between 0.05 mW·mm⁻² and 280 mW·mm⁻².

Energy flux density of the electromagnetic field in noncontact mode where electrode is spaced from the patient body by spacing object with beneficial dielectric parameters e.g.: bolus filled with water, silicon spacing object and/or others) may be preferably in the range between 0.01 mW·mm⁻² and 500 mW·mm⁻², more preferably in the range between 0.01 mW·mm⁻² and 240 mW·mm⁻² or even more preferably in the range between 0.01 mW·mm⁻² and 60 mW·mm⁻² or the most preferably in the range between 0.05 mW·mm⁻² and 12 mW·mm⁻².

Therapies provided during the treatment may be applied sequentially, simultaneously and/or with some overlay.

Shock waves are mechanical waves characterized steep pressure amplitude growth in comparison to the surrounding pressure. The shock waves are characterized by non-linearity during the wave propagation. The positive peak pressure is above 0.1 MPa, more preferably 3 MPa, even more preferably at least 7 MPa, most preferably at least 15 MPa. The peak pressure in the positive maximum may be up to 150 MPa. The pulse duration of the shock wave (based on the time the pressure exceeds a half value of peak positive pressure) may be preferably in the range of hundreds of nanoseconds to tens of microseconds.

In still another embodiment the mechanical shock waves may be used in order to achieve the better results in the soft tissue. The energy flux density of the shock waves is preferably in the range between 0.001 mW·mm⁻² and 160 mW·mm⁻², more preferably in the range between 0.005 mW·mm⁻² and 100 mW·mm⁻², most preferably in the range between 0.001 mW·mm⁻² and 50 mW·mm⁻². Shock wave pulses may be applied in frequency range between 0.1 Hz or less to 100 Hz or more preferably 0.5 Hz to 50 Hz or the most preferably between 1 Hz to 30 Hz.

Shock wave treatment energy source may also be used adjacent to the patient soft tissue in direct, indirect and/or no-contact with the patient's skin.

Still another embodiment combining plasma and massage and/or other treatment therapy (e.g. removing cellulite or rejuvenation by RF treatment energy source) may be used to provide better therapy and improve skin rejuvenation, improve blood and lymph stimulation, help to treat scars and/or skin injuries, skin illnesses (e.g. lupus, acne, fungal infections, influence vitiligo in positive manner and/or other), or disinfection/sterilization of the patient surface.

Plasma therapy may influence conductivity of the patient skin surface that may help to improve delivering of the RF signal into the patient soft tissue. Plasma may be also used in order to improve focusing of the RF signal during the noncontact method because of plasma conductive properties and patient surface ionization.

RF in combination with plasma may have the added benefit that RF electrodes may help to ionize gas and simplify of plasma creation and/or regulation of plasma parameters. Also light therapy may influence ionization of an air/gas between patient surface and light source and therefore influence plasma parameters in a defined manner and/or simplify of plasma creation.

The gas used for generation of plasma may be single type of gas e.g. argon, helium, nitrogen, oxygen. The gas may be also mixture of single types of gas e.g. air, mixture of argon and helium, argon and hydrogen, argon and oxygen, oxygen and hydrogen, nitrogen and oxygen, argon and oxygen or argon and oxygen together with nitric oxide. Oxygen may be used in order to produce plasma containing ozone and other reactive allotropes of oxygen e.g. tetraoxygen etc. The generated plasma may also interact with pure oxygen to create ozone. Plasma may also be doped by above mentioned gasses during the therapy.

Temperature of the plasma may be between 18° C. to 65° C. or in more preferred embodiment between 25° C. to 62° C. or in even more preferred embodiment between 32° C. to 60° C. or in the most preferred embodiment between 35° C. to 38° C. Plasma may be applied in range between 1 s to 60 min or in more preferred protocols in range between 10 s to 40 min or in the most preferred protocols in range between 30 s to 30 min. Plasma may also be used in a pulse mode that lasts between 0.1 s to at least 30 s or in more preferred treatment protocols between 0.1 s to 20 s or in the most preferred embodiment between 2 s to 10 s.

Plasma may be produced in different ways. The most preferred embodiment produces plasma by voltage between electrodes in range between 100 V to 30 kV in more preferred embodiment between 1 kV to 30 kV in the most preferred embodiment in range between 1 kV to 20 kV. Plasma generation may use an electrode frequency of 20 kHz to 27 MHz in more preferred embodiment between 0.8 MHz to 15 MHz or in the most preferred embodiment between 1 MHz to 14 MHz. Plasmas as described in U.S. Provisional Application No. 62/409,665, incorporated herein by reference, may also be used.

Light therapy may be combined with massage therapy and/or any other treatment therapy. The light may be coherent and/or non-coherent light. Most preferably, the electromagnetic waves include wavelengths ranging from 405 to 1500 nm or more preferably ranging from 600 nm to 1500 nm or the most preferably ranging from 760 nm to 1200 nm. Wave length in the range of infrared light is very effective for heating of the soft tissue layers located beneath the skin surface. Electro-magnetic waves (RF waves and/or optical waves) may also be polarized to improve treatment results and/or improve safety of the patient as described in U.S. Provisional Application No. 62/331,072.

Light therapy may be used in combination with massage for preheating the layers of soft tissue, may influence cell metabolism stimulation or may provide a bio-stimulation effect. Therapy by light may also be used separately and may be targeted to several specific pigment targets and/or may be focused to influence collagen fibers, adipose tissue and/or other layers of the soft tissue.

Treatment by light energy source may also be very effective for wrinkle reduction, removing of skin diseases, pigment imperfections and/or others. Treatment by lighting combination with massage therapy may accelerate healing process of damaged tissue and make the treatment more comfortable and improve treatment results.

A light treatment energy source may be adjacent to the patient's soft tissue or in direct contact with the patient surface, indirect may be spaced from patient's surface by any material except air gap and/or no-contact spaced from patient's skin by air gap. In indirect contact spacing material may be transparent, translucent layer (e.g. glass or crystal with specific optical properties, liquid solution that may include specific active substances that may modify light parameters and/or soft tissue reaction to the delivered light energy). In some embodiment other material that separates the light treatment energy source from the patient's surface may be used, and may not be translucent, with a majority of visible wavelengths but may have low absorbance for infrared wavelengths.

Light therapy may be applied as continual radiation lasting between 2 seconds to at least 40 minutes or in a more preferred embodiment between 2 seconds to 30 minutes or in the most preferred embodiment between 2 seconds to 15 minutes.

The energy flux density of light therapy during the continual mode may be in range between 0.05 mW·mm⁻² to 1.2 W·mm⁻², more preferably in the range of 0.05 mW·mm⁻² to 0.63 W·mm⁻², even more preferably in the range of 0.05 mW·mm⁻² to 0.4 W·mm⁻², most preferably in the range of 0.05 mW·mm⁻² to 0.2 W·mm⁻².

Light therapy may be applied in pulsed mode as well. The pulses may last between 50 μs to 1 s, in a more preferred protocol pulses may last between 1 ms to 1 s, in the most preferred embodiment pulses may last between 1 ms to 52 ms. Pauses between two pulses may last between 50 μs to 1 s or in a more preferred protocol between 1 ms to 1 s or in the most preferred protocol between 1 ms to 45 ms.

The flux density of light therapy during the continual mode may be in range between 0.05 mW·mm⁻² to 13 W·mm⁻², more preferably in the range of 0.05 mW·mm⁻² to 6 W·mm⁻², even more preferably in the range of 0.05 mW·mm⁻² to 2 W·mm⁻², most preferably in the range of 0.05 mW·mm⁻² to 0.6 W·mm⁻².

The sum of energy flux density of the radio frequency waves and the optical waves applied to the patient during the therapy, where the therapy means simultaneous, successive or overlap treatment or treatments, may last up to 120 minutes, more preferably up to 60 minutes, most preferably up to 30 minutes, is in the range of 0.03 mW·mm⁻² and 1.2 W·mm⁻², more preferably in the range of 0.05 mW·mm⁻² and 0.9 W·mm⁻², most preferably in the range of 0.01 mW·mm⁻² and 0.6 W·mm⁻². The energy flux density of optical waves constitutes at least 1%, more preferably at least 3% and most preferably at least 5% of the sum of energy flux density.

Preheating and/or precooling may be provided in the first and/or the last treatment therapy. Preheating and/or precooling is not combined with any other treatment therapy preheating or precooling of the soft tissue may influence dielectric properties of the soft tissue, soft tissue laxity, may prevent hot spots, may also influence pain receptors (shift patient pain threshold) and/or others.

Preheating or precooling by at least one treatment energy source of the patient's soft tissue may be done in temperature range from 25° C. to 60° C. or in range from 32° C. to 50° C. or in range from 36° C. to 45° C. or in range from 36° C. to 42° C. or in range from 36° C. to 38° C.

Heating or cooling of the soft tissue during the treatment may be provided by any treatment energy source (e.g. RF adipose tissue removing) and may be combined with one or more other treatment therapies (e.g. cellulite removing treatment therapy, massage treatment therapy, light rejuvenation treatment therapy and/or other).

Heating or cooling by at least one treatment energy source of the patient's soft tissue may be done in temperature range from 25° C. to 60° C. or in range from 32° C. to 50° C. or in range from 36° C. to 45° C.

A cooling/heating mechanism may be used. In some embodiments air may be blown on the patient skin and/or sucked from the patient skin and/or on a protecting layer in order to control surface temperature, create a temperature gradient in the patient soft tissue, cool treatment energy sources (e.g. RF electrodes), cool the patient surface, provide micro lymph drainage, remove moisture and/or make the treatment more comfortable. A cooling/heating function may be also provided by flow of a liquid, by thermal diffusion provided through solid, liquid, gel, gaseous material with good thermal conductivity and/or by thermoelectric method based on the Peltier effect.

The ranges of described parameters in this document are just non-limited examples of possible ranges that may be used.

Treatment sources of energy (e.g. included in massage units and/or other treatment units) may be located inside of the treatment sleeve, between layers of the treatment sleeve and/or outside of the treatment sleeve. Outside of the treatment sleeve may be below the treatment sleeve between treatment sleeve and patient body or externally of the treatment sleeve where any layer of the treatment sleeve is closer to the patient surface than treatment energy source. One or more treatment energy sources may produce energy to the patient soft tissue from the outside of the treatment sleeve without contact with the treatment sleeve and/or in contact with the treatment sleeve. A treatment energy source may be located in the treatment sleeve and may be separated from the patient's surface by spacing object described in U.S. Provisional Application No. 62/331,072, incorporated herein by reference.

The treatment energy sources (located inside the treatment sleeve) may be placed in the pressure cell/massage object, between pressure cells/massage objects, above the pressure cell/massage object and/or under pressure cell/massage object. In other embodiments the treatment energy sources may be placed next to each other and may create an arbitrary pattern.

In an embodiment for systematic lymph drainage by applying pressure and massage in combination with heating the patient soft tissue by RF, adjustable electrodes may be used, as described in U.S. Provisional Application No. 62/351,156, incorporated herein by reference.

In some embodiments the treatment effect may be improved by applying drugs before, during and/or after treatment as described above and in U.S. Provisional Application No. 62/331,060 incorporated herein reference. Also, injection of an electrically insulating fluid, such as Dextran™ (complex branched glucan), may be used to absorb microwave energy and protect tissues beyond the target zone. Element changing pressure value means an element which changes the pressure applied to the patient.

Thus, novel devices and methods have been shown and described. Various changes and substitutions may of course be made without departing from the spirit and scope of the invention. The invention, therefore, should not be limited, except by the following claims, and their equivalents.

1. A method of soft tissue treatment of a patient comprising:
placing a treatment sleeve with at least one first treatment energy source onto the patient;
providing massage of the patient's soft tissue by at least one massage unit, with the massage unit including at least two elements changing pressure value applied on the patient's soft tissue;
and providing different treatment therapy than massage by a second treatment energy source;
wherein the at least one massage unit creates a pressure gradient between at least two successive elements changing pressure value in range from 0% to 95% on the patient's soft tissue across a treated body area and stimulates body liquid flow.

2. The method according to claim 1 using a successive therapy speed in a range between 0.1 cm·s⁻¹ and 50 cm·s⁻¹.

3. The method according to claim 1 wherein a temperature of the patient's soft tissue is maintained during the treatment in range between 25° C. to 60° C.

4. The method according to claim 1 further including providing the treatment in a pattern.

5. The method according to claim 4 wherein the treatment in a pattern is under automatic control and providing treatment without a human operator.

6. The method according to claim 4 wherein the pattern simulates linear or curvilinear movement or combinations of curvilinear with linear moves.

7. The method according to claim 1 with each element changing pressure value between 1 cm² to 600 cm².

8. The method according to claim 1 with the each element changing pressure value applying pressure on the soft tissue in a range between from 10 Pa to 300,000 Pa.

9. The method according to claim 1 with the second treatment energy source inside of the treatment sleeve.

10. The method according to claim 1 with the second treatment energy source outside of the treatment sleeve

11. The method according to claim 1 wherein the second treatment energy source provides treatment by generated magnetic field.

12. The method according to claim 1 wherein the second treatment energy source provides treatment by electric current.

13. The method according to claim 1 wherein the second treatment energy source provides treatment using acoustic waves.

14. The method according to claim 1 wherein the second treatment energy source provides treatment using plasma.

15. The method according to claim 1 with the soft tissue treated covering an area during one or more treatment in a range between 1 cm² to 3 m².

16. The method according claim 1 wherein massage effect is provided by acoustic wave with an energy flux density between 0.001 mW/mm² to 160 mW/mm².

17. A method of soft tissue treatment of a patient comprising: placing a treatment sleeve with at least one massage unit adjacent to patient's soft tissue and providing lymph drainage of the patient's soft tissue;
and providing a light therapy of the patient's soft tissue by a light treatment energy source;
with the massage unit creating a pressure gradient on the patient's soft tissue across a treated area and stimulating body liquid flow.

18. The method according to claim 17 with an energy flux density of light treatment energy in a range between 0.05 mW·mm⁻² to 13 W·mm⁻².

19. A method of soft tissue treatment of a patient comprising: placing a treatment sleeve with at least one massage unit adjacent to a patient's soft tissue and providing lymph drainage of the patient's soft tissue; and providing a ultrasound treatment of the patient's soft tissue by an ultrasound treatment energy source;
with the massage unit creating a pressure gradient on the patient's soft tissue across a treated area and stimulating body liquid flow.

20. The method according to claim 19 with an energy flux density of ultrasound treatment energy source in range between 0.001 mW·mm⁻² to 224 W·mm⁻².

21. A method for soft tissue treatment of a patient comprising: placing a treatment sleeve with at least one massage unit adjacent to patient's soft tissue to provide lymph drainage of the patient's soft tissue; and providing a RF treatment of the patient's tissue by a RF treatment energy source;
with the massage unit creating a pressure gradient on the patient's soft tissue across a treated area and stimulating a body liquid flow.

22. The method according to claim 21 with an automatic control and providing treatment without a human operator.

23. The method according to claim 21 with the RF treatment energy source providing RF energy in range between 90 kHz to 40.7 MHz.

24. The method according to claim 23 with an energy flux density of the RF treatment energy source in the range between 0.01 mW·mm−2 and 500 mW·mm−2.

25. The method according to claim 21 with the treatment sleeve adapted to encircle at least part of the patient.

26. The method according to claim 25 with the treatment sleeve extendable by using one or more treatment pieces.

27. The method according to claim 25 providing treatment of multiple arbitrary body area at the same time.

28. The method according to claim 21 further including automated moving with the treatment energy source according to pattern.

29. The method according to claim 21 with the massage unit including at least two elements changing pressure value applying the pressure on the soft tissue.

30. The method according to claim 29 with the massage unit including at least two pressure cell.
